(19) United States
(12) Patent Application Publication
(10) Pub. No.: US 2023/0210830 A1
(43) Pub. Date: Jul. 6, 2023

(54) STABLE PHARMACEUTICAL COMPOSITION OF NIMODIPINE

(71) Applicant: ALKEM LABORATORIES LIMITED, Maharashtra (IN)

(72) Inventors: Arun Kumar PANDEY, Maharashtra (IN); Umadoss POTHUVAN, Maharashtra (IN); Ganesh Shashikant KHAMKAR, Maharashtra (IN); Ulhas DHUPPAD, Maharashtra (IN)

(21) Appl. No.: 18/090,024

(22) Filed: Dec. 28, 2022

(30) Foreign Application Priority Data
Jan. 1, 2022 (IN) 202221000027

(51) Int. Cl. A61K 31/4422; A61K 9/00; A61K 47/10; A61K 9/08; A61K 47/14

(52) U.S. Cl. CPC A61K 31/4422; A61K 9/0053; A61K 47/10; A61K 9/08; A61K 47/14

(57) ABSTRACT

The present invention relates to a stable aqueous solution composition suitable for oral administration comprising nimodipine, polyethylene glycol, dehydrated alcohol, glycerin, water and preservative.

### FIELD OF THE INVENTION

The present invention relates to a stable aqueous solution composition of nimodipine and process for the preparation thereof. This invention more particularly relates to a stable aqueous pharmaceutical composition of nimodipine for oral administration and process for the preparation thereof.

### BACKGROUND OF THE INVENTION

Nimodipine is a 1,4-dihydropyridine calcium channel blocker. Chemically, nimodipine is isopropyl 2-methoxyethyl 1,4-dihydro-2,6-dimethyl-4-(m-nitrophenyl)-3,5-pyridinedicarboxylate.

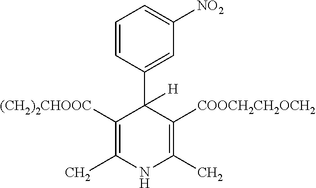

The molecular weight of nimodipine is 418.5 g/mol, and the molecular formula is C₂₁H₂₆N₂O₇. The octanol:water partition coefficient, log P value is 3.05. The pKa values are 5.41 (Strongest Basic). Like many other 1,4-dihydropyridines, nimodipine is insoluble in water, but soluble in ethanol, polyethylene glycol 400, dimethyl sulfoxide (DMSO), and chloroform. Nimodipine is sensitive to light, although substantially less so than nifedipine; the half-life of nimodipine solution under fluorescent laboratory light is approximately 7 Hr. Under diffuse daylight, the half-life of nimodipine solution is substantially prolonged, and under yellow sodium-vapor light nimodipine is essentially stable.

Nimodipine is a 1,4-dihydropyridine calcium-channel antagonist and binds specifically to L-type voltage-gated calcium channels, is considered the standard of care for the prevention of vasospasm in patients who have suffered subarachnoid hemorrhage. The usual dosage is 60 mg orally every four hours for 21 days; however, a decreased dosage and increased frequency of administration to 30 mg every two hours or 15 mg every hour may be required for some patients with hypotension. Nimodipine has a high first-pass effect, and its bioavailability in humans is estimated as 12%. It is well absorbed after oral administration and its t_{1/2} in humans is estimated to be 90 min. Nimodipine is rapidly metabolized, and its metabolites are excreted largely in bile.

Nimodipine formulations are available in the United States of America under the brand name of NYMALIZE® and NIMOTOP®. The NYMALIZE® 3 mg/mL and 6 mg/mL oral solution products and NIMOTOP® Capsule 30 mg product is indicated for the improvement of neurological outcome by reducing the incidence and severity of ischemic deficits in adult patients with subarachnoid hemorrhage (SAH) from ruptured intracranial berry aneurysms regardless of their post-ictus neurological condition (i.e., Hunt and Hess Grades I-V). It is available in two strengths i.e. 3 mg/mL (Discontinued from market) and 6 mg/mL. The NYMALIZE® 3 mg/mL contains ethanol, glycerin, methylparaben, polyethylene glycol, sodium phosphate monobasic, sodium phosphate dibasic, and water. Further the NYMALIZE® 6 mg/mL contains ethanol, glycerin, methylparaben and polyethylene glycol 400. The NIMOTOP® liquid filled capsule contains 30 mg of nimodipine in a vehicle of glycerin, peppermint oil, purified water and polyethylene glycol 400. The soft gelatin capsule shell contains gelatin, glycerin, purified water and titanium dioxide.

Nimodipine formulation is also available in the Europe as NIMOTOP® 30 mg film-coated tablet and NIMOTOP® 0.02% Solution for Infusion and it is indicated for the prevention or treatment of ischaemic neurological deficits following aneurysmal subarachnoid haemorrhage. Each NIMOTOP® 30 mg film-coated tablet contains microcrystalline cellulose, maize starch, povidone, crospovidone, magnesium stearate, hypromellose, macrogol 4000, titanium dioxide E171, iron oxide yellow E172. Further each NIMOTOP® 0.02% Solution for Infusion contains Ethanol 96%, Macrogol 400, sodium citrate, citric acid, Water for Injections Ph. Eur.

Nimodipine is used for the oral treatment for the improvement of neurological outcome by reducing the incidence and severity of ischemic deficits in adult patients with subarachnoid hemorrhage (SAH) from ruptured intracranial berry aneurysms regardless of their post-ictus neurological condition (i.e., Hunt and Hess Grades I-V) in the form of either 3 mg/mL, 6 mg/mL oral solution or 30 mg capsule, 30 mg tablet and 0.02% solution for infusion. Nimodipine is a dihydropyridine calcium channel blocker. The contractile processes of smooth muscle cells are dependent upon calcium ions, which enter these cells during depolarization as slow ionic transmembrane currents. Nimodipine inhibits calcium ion transfer into these cells and thus inhibits contractions of vascular smooth muscle.

Green, A. et. al. in American Journal of Health-System Pharmacy, 61:1493-6 (2004) discloses the stability of nimodipine withdrawn from nimodipine capsules and stored in amber-tinted oral syringes and placed in light-protected bags, for up to 31 days under various storage conditions.

AlSheyyab et. al. in Journal of Applied Pharmaceutical Science, Vol. 9(09), pp 030-037 (September 2019) discloses the methods to enhance the water solubility of nimodipine, a hydrophobic drug, using a solid dispersion (SD) technique. The use of Soluplus® with nimodipine to prepare solid dispersions found to be a promising technique to enhance both nimodipine solubility and the dissolution profile. Soluplus® as a novel hydrophilic polymeric carrier was used. This enhancement was more pronounced in the dispersions prepared using the supercritical fluid technique.

Ehrlich et. al. in vivo 33: 1967-1975 (2019) discloses that the oral nimodipine is administered to improve clinical outcome in patients with aneurysmal subarachnoid hemorrhage (aSAH). In this study, clinical outcome in patients with and without oral nimodipine administration was assessed and concluded that the oral nimodipine administration improved clinical outcome of patients after aSAH and should be administered routinely for such patients.

Prajapat et. al. in Journal of Drug Delivery Science and Technology, 53 (2019) discloses the solubility enhancement as well as percent drug release of BCS class II drug, nimodipine using liquisolid technique. Compatibility and potential drug substance relationship with excipients were explored using Differential scanning calorimetry (DSC), X-ray diffraction (XRD), Fourier transform infrared spectroscopy (FTIR) and Scanning electron microscopy (SEM). A percentage of drug release was also determined for discrimination in compendial media and biorelevant media. Studies of pharmacodynamics and pharmacokinetics studies in rats have also been conducted to predict the formulation's in vivo behavior.

Prajapat et. al. in Journal of Drug Delivery Science and Technology, 39 (2017) discloses the Formulation and evaluation of self-emulsifying drug delivery system (SMEDDS) for nimodipine, a BCS class II drug. In SMEDDS, the excipients are selected based on higher solubility due to deliver drug in solubilized state in GIT as well as to reduce volume of formulation to be administer.

Zhao et. al. in Asian Journal of Pharmaceutical Sciences, 9 (2014) discloses the solid dispersion (SD) in the development of a nimodipine delayed-release tablet formulation in that development of a nimodipine delayed-release tablet formulation using SD. The dissolution rate and solubility of nimodipine solid dispersions were improved compared with the pure drug.

Barmpalexis et. al. in Journal of Pharmaceutical and Biomedical Analysis, 49 (2009) discloses an efficient isocratic reversed-phase high-performance liquid chromatography method which was developed, optimized and validated to separate the calcium antagonist nimodipine and its impurities (A, B and C) using statistical experimental design.

U.S. Pat. No. 4,537,898 discloses liquid formulations of dihydropyridine comprising, by weight, about 1 to 5 parts of a dihydropyridine selected from the group consisting of nisoldipine, nimodipine and 3-methyl 5-trifluoroethyl diester of 4-(2-chlorophenyl)-2,6-dimethyl-1,4-dihydropyridine-3,5-dicarboxylic acid, 30 to 50 parts of weight of a solubilizing agent comprising glycerol polyethylene glycol oxystearate oxyethylated with about 35, 45 or 60 mols of ethylene oxide and 70 to 50 parts of a diluent consisting essentially of water, ethanol, glycerol, 1,2-propylene glycol, polyethylene glycol 200, polyethylene glycol 400 or polyethylene glycol 600.

U.S. Pat. No. 4,857,312 discloses 40 gm of nimodipine are dissolved in 500 gm of ethanol and 200 gm of polyethylene glycol 400. 8 gm of polyvinylpyrrolidine 25 are dissolved separately in 83 gm of water and the solution is then added to the nimodipine solution. 40 gm of anhydrous glycerol and 2 gm of peppermint oil are then added. The solution is filtered and bottled.

U.S. Pat. No. 5,114,956 discloses method of making a parenteral formulation consisting essentially of about a) 0.01-0.4% by weight of nimodipine, relative to 100 parts by weight of a solvent consisting essentially of about b) 15-40% by weight of polyethylene glycol, 15-30% by weight of ethanol, and water, which comprises dissolving the nimodipine in the polyethylene glycol and ethanol, and then adding the water to the solution.

U.S. Pat. No. 9,339,553 discloses a liquid composition of an insoluble medicament, said liquid composition containing insoluble medicament, oil for injection, phospholipid, and solvent, with the following percentage by weight for each component: insoluble medicament 0.01-10%, oil for injection 0%-20%, phospholipid 20-45%, and solvent 20-89%; wherein the liquid composition of an insoluble medicament does not contain surfactant of polyoxyethylene castor oil or polysorbate; wherein the liquid composition is a clear, transparent solution and does not contain water, and wherein the liquid composition is stable within 8 hours after being hydrated with injectable solvents.

U.S. Pat. No. 8,486,374 discloses a waterless composition suitable for delivery of an active agent to a body surface or cavity comprising: a vehicle comprising: a) about 70% to about 96.5% by weight of a mixture of PEG 200 and PEG 400; b) about 0.01% to about 10% of at least one surface active agent and/or about 0.01% to about 5% of a polymeric agent; c) about 0% to about 30% of a secondary hydrophilic solvent; and d) about 0% to about 5% of a silicone oil; and e) about 3% to about 25% hydrophobic propellant; wherein the composition is otherwise substantially free of a hydrophobic solvent; and wherein the vehicle and the propellant are sufficiently miscible that the components may be homogeneously distributed with mild shaking.

US20070117851 discloses liquid pharmaceutical composition comprising: (a) nimodipine; (b) an alcohol; and (c) a solvent; with the proviso that if said alcohol comprises ethanol, the ethanol concentration of said pharmaceutical composition is less than about 15 percent by weight. Further discloses novel liquid pharmaceutical compositions of nimodipine with improved properties such as higher concentrations of nimodipine with lower concentrations of ethanol than that in the art which makes it more suitable for administration to patients via an intraoral or intranasal tube.

Despite the above mentioned prior art disclosing various pharmaceutical formulations of nimodipine comprising using solvents or co-solvents, preservative and one or more excipients, there is still exists a need for a stable aqueous pharmaceutical compositions of nimodipine suitable for oral administration and processes for the preparation thereof which is able to overcome the problem associated with the existing formulation such as solubility, stability and impurity, while the most one difficult being a selection of excipients for the development of a stable pharmaceutical compositions of nimodipine for oral solution. The commercially available marketed product NYMALIZE® 6 mg/mL is a non-aqueous composition, which does not contain water in the composition. However, the NYMALIZE® 3 mg/mL aqueous oral solution which contain water degrades more as compare to NYMALIZE® 6 mg/mL non-aqueous composition during the shelf life of product. It has now been found surprisingly that a stable aqueous solution composition of nimodipine suitable for oral administration could be prepared and the compositions simultaneously achieve desired stability characteristic and dissolution profile.

### OBJECT OF THE INVENTION

It is an object of the present invention to provide a stable aqueous pharmaceutical composition of nimodipine suitable for oral administration.

It is another object of the present invention to provide a stable aqueous composition suitable for oral administration comprising:
a) Nimodipine;
b) a pharmaceutically acceptable solvent system consisting of Polyethylene glycol and alcohol;
c) a pharmaceutically acceptable Co-solvent;
d) water; and
e) one or more pharmaceutically acceptable excipients.
   It is another object of the present invention to provide a stable aqueous solution composition suitable for oral administration consisting of:
a. Nimodipine at a concentration of 6 mg/ml;
b. a pharmaceutic ally acceptable solvent system consisting of Polyethylene glycol and alcohol in a concentration of about 670 mg/ml and about 4 mg/ml respectively;
c. glycerin at a concentration of about 480 mg/ml;
d. water at a concentration of about 25 mg/ml; and
e. methyl paraben at a concentration of about 3 mg/ml.
   It is another object of the present invention to provide a stable aqueous solution composition suitable for oral administration consisting of:
a. Nimodipine at a concentration of about 6 mg/ml to about 12 mg/ml;
b. a pharmaceutically acceptable solvent system consisting of Polyethylene glycol and alcohol in a concentration of about 500 mg/ml to about 700 mg/ml and about 1 mg/ml to about 5 mg/ml respectively;
c. glycerin at a concentration of about 400 mg/ml to about 500 mg/ml;
d. water at a concentration of about 20 mg/ml to about 50 mg/ml; and
e. methyl paraben at a concentration of about 1 mg/ml to about 5 mg/ml.
   It is another object of the present invention to provide a stable aqueous solution composition suitable for oral administration consisting of:
a. Nimodipine at a concentration of about 0.3% w/v to about 1.2% w/v;
b. a pharmaceutically acceptable solvent system consisting of Polyethylene glycol and alcohol in a concentration of about 50% w/v to about 70% w/v and about 0.1% w/v to about 0.5% w/v respectively;
c. glycerin at a concentration of about 40% w/v to about 50% w/v;
d. water at a concentration of about 2% w/v to about 5% w/v; and
e. methyl paraben at a concentration of about 0.1% w/v to about 0.5% w/v.
   It is another object of the present invention to provide a stable aqueous solution composition suitable for oral administration consisting of:
a. Nimodipine at a concentration of about 0.3% v/v to about 1.2% v/v;
b. a pharmaceutically acceptable solvent system consisting of Polyethylene glycol and alcohol in a concentration of about 50% v/v to about 70% v/v and about 0.1% v/v to about 0.5% v/v respectively;
c. glycerin at a concentration of about 35% v/v to about 40% v/v;
d. water at a concentration of about 2% v/v to about 5% v/v;
e. methyl paraben at a concentration of about 0.1% v/v to about 0.5% v/v.
   It is another object of the present invention to provide a stable aqueous solution composition suitable for oral administration consisting of:
a. Nimodipine at a concentration of about 0.3% w/w to about 1.2% w/w;
b. a pharmaceutically acceptable solvent system consisting of Polyethylene glycol and alcohol in a concentration of about 50% w/w to about 70% w/w and about 0.1% w/w to about 0.5% w/w respectively;
c. glycerin at a concentration of about 35% w/w to about 45% w/w;
d. water at a concentration of about 1.7% w/w to about 4% w/w;
e. methyl paraben at a concentration of about 0.1% w/w to about 0.5% w/w.
   It is also an object of the present invention to provide a process to prepare a stable aqueous pharmaceutical composition for oral administration comprising:
a) Nimodipine;
b) Polyethylene glycol and alcohol as solvent;
c) glycerin;
d) purified water; and
e) methyl paraben.
   Wherein the process comprises:
(a) Preparation of glycerin solution by heating at 80-90° C. and adding methyl paraben under continuous stirring to get clear solution;
(b) Preparation of nimodipine solution by using PEG-400 and ethanol as solvent;
(c) Mixing solution (b) into solution (a) with addition of water;
(d) Filter the bulk solution (c) through 3.0μ (Polypropylene) filter; and
(e) Transferring the filtered solution into a storage vessel.

### SUMMARY OF THE INVENTION

The present invention provides a stable aqueous pharmaceutical composition of nimodipine and process for the preparation thereof.

In one aspect of the present invention, there is provided a stable aqueous pharmaceutical composition of nimodipine in a liquid solution suitable for oral administration.

In another aspect of the present invention, there is provided a stable aqueous pharmaceutical composition of nimodipine consisting of:

a) Nimodipine, as an active ingredient;

b) Polyethylene glycol and alcohol as solvent;

c) Co-solvent;

d) purified water; and

e) one or more pharmaceutically acceptable excipient.

In another aspect of the present invention, there is provided a stable aqueous solution composition suitable for oral administration consisting of:
a. Nimodipine at a concentration of 6 mg/ml;
b. a pharmaceutic ally acceptable solvent system consisting of Polyethylene glycol and alcohol in a concentration of about 670 mg/ml and about 4 mg/ml respectively;
c. glycerin at a concentration of about 480 mg/ml;
d. water at a concentration of about 25 mg/ml; and
e. methyl paraben at a concentration of about 3 mg/ml.
   In another aspect of the present invention, there is provided a stable aqueous solution composition suitable for oral administration consisting of:
a. Nimodipine at a concentration of about 6 mg/ml to about 12 mg/ml;
b. a pharmaceutically acceptable solvent system consisting of Polyethylene glycol and alcohol in a concentration of about 500 mg/ml to about 700 mg/ml and about 1 mg/ml to about 5 mg/ml respectively;
c. glycerin at a concentration of about 400 mg/ml to about 500 mg/ml;
d. water at a concentration of about 20 mg/ml to about 50 mg/ml; and
e. methyl paraben at a concentration of about 1 mg/ml to about 5 mg/ml.
   In further aspect of the present invention, there is provided a stable aqueous solution composition suitable for oral administration consisting of:
a. Nimodipine at a concentration of about 0.3% w/v to about 1.2% w/v;
b. a pharmaceutically acceptable solvent system consisting of Polyethylene glycol and alcohol in a concentration of about 50% w/v to about 70% w/v and about 0.1% w/v to about 0.5% w/v respectively;
c. glycerin at a concentration of about 40% w/v to about 50% w/v;
d. water at a concentration of about 2% w/v to about 5% w/v; and
e. methyl paraben at a concentration of about 0.1% w/v to about 0.5% w/v.
   In further aspect of the present invention, there is provided a stable aqueous solution composition suitable for oral administration consisting of:
a. Nimodipine at a concentration of about 0.3% v/v to about 1.2% v/v;
b. a pharmaceutically acceptable solvent system consisting of Polyethylene glycol and alcohol in a concentration of about 50% v/v to about 70% v/v and about 0.1% v/v to about 0.5% v/v respectively;
c. glycerin at a concentration of about 35% v/v to about 40% v/v;
d. water at a concentration of about 2% v/v to about 5% v/v;
e. methyl paraben at a concentration of about 0.1% v/v to about 0.5% v/v.
   In further aspect of the present invention, there is provided a stable aqueous solution composition suitable for oral administration consisting of:
a. Nimodipine at a concentration of about 0.3% w/w to about 1.2% w/w;
b. a pharmaceutically acceptable solvent system consisting of Polyethylene glycol and alcohol in a concentration of about 50% w/w to about 70% w/w and about 0.1% w/w to about 0.5% w/w respectively;
c. glycerin at a concentration of about 35% w/w to about 45% w/w;
d. water at a concentration of about 1.7% w/w to about 4% w/w;
e. methyl paraben at a concentration of about 0.1% w/w to about 0.5% w/w.
   In another aspect of the present invention, there is provided a stable aqueous pharmaceutical composition of nimodipine and process preparing thereof, comprising
a) Nimodipine;
b) Polyethylene glycol and alcohol as solvent;
c) glycerin;
d) purified water; and
e) methyl paraben;
   Wherein the process comprises:
a. Preparation of glycerin solution by heating at 80-90° C. and adding methyl paraben under continuous stirring to get clear solution;
b. Preparation of nimodipine solution by using PEG-400 and ethanol as solvent;
c. Mixing solution (b) into solution (a) with addition of water;
d. Filtering the bulk solution (c) through 3.0μ (Polypropylene) filter; and
e. transferring the filtered solution into storage vessel.

### DETAILED DESCRIPTION OF THE INVENTION

Before the present process and methods are described, it is to be understood that this invention is not limited to particular compounds, formulas or steps described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges also encompassed within the invention, subject to any specifically excluded limit in the stated range. Where the stated range includes one or both of the limits, ranges excluding either both of those included limits are also included in the invention.

Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a compound” includes a plurality of such compounds and reference to “the step” includes reference to one or more step and equivalents thereof known to those skilled in the art, and so forth.

The publications discussed herein are provided solely for their availability to the applicant prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “stable” as used herein refers to chemical stability of nimodipine in aqueous Oral liquid dosage form wherein the total impurity formed is less than 2%, when the dosage form is subjected to the stability conditions of 25° C./60% RH, 30° C./65% RH and 40° C./75% RH for at least 3 months.

The term “composition” as used herein is intended to encompass a product comprising the specified ingredients (and in the specified amount, if indicated), as well as any product which results, directly or indirectly, from combination of the specified ingredients in the specified amounts. By “pharmaceutically acceptable” it is mean that the diluent, excipient or carrier must be compatible with the other ingredients of the formulation and not deleterious to the recipient thereof.

As term “active ingredient” as used herein means an ingredient or compound having an intended biological effect. “Active ingredient” may be broadly construed to include an active compound and vice versa. Such active ingredients or active compounds are thus considered to be “biologically active”.

The term “solvent” as used herein refers a solvent that is a substance that dissolves a solute, resulting in a solution. A solvent is usually a liquid but can also be a solid, a gas, or a supercritical fluid. Water and alcohol mostly used as solvent in the formulation. For purposes of this invention, the term “dehydrated alcohol” is used interchangeably with the term “absolute alcohol” and “alcohol”. The amount of dehydrated alcohol in a particular formulation will vary based on the intended formulation and the solubility of the nimodipine. The amount of dehydrated alcohol in the formulations of the present invention can range from about 0.10% w/v to about 0.50% w/v, more preferably from about 0.2% w/v to about 0.45% w/v.

The term “co-solvent,” as used herein, refers to a compound having the ability to increase the solubility of a solute. Co-solvents are added to increase the solvent power of the primary substance in the mixture. The advantage of cosolvent technology enhancing drug solubility in a liquid-based formulation.

In an embodiment, the present invention provides that the one or more pharmaceutically acceptable excipient(s) constituting the disclosed stable pharmaceutical compositions of nimodipine are solvents or co-solvents, preservative and others.

In another embodiment, solvent or solubilizing agents used in the composition of the present invention is selected from but are not limited to, ethanol or alcohol, propanol, isopropanol, propylene glycol, polyethylene glycol of various molecular weights, dichloromethane, dimethylisosorbide, ethyl lactate, N-methylpyrrolidones, glycofurol, decaglycerol mono-, dioleate, triglycerol monooleate, polyglycerol oleate, mixed diesters of Caprylic/Capric acid and propylene glycol, ethyl oleate, glyceryl monooleate, Vitamin E TPGS, alpha tocopherol, or mixtures thereof. The preferred solvent or solubilizing agents is polyethylene glycol 400 and ethanol. The amount of PEG 400 may be present from about 50% w/v to about 70% w/v, preferably be present from about 55% w/v to about 65% w/v. The amount of ethanol or alcohol may be present from about 0.1% w/v to about 0.5% w/v, more preferably from about 0.2% w/v to about 0.4% w/v.

In another embodiment, co-solvents used in the composition of the present invention are selected from but are not limited to, glycerol or glycerin, dimethylsulfoxide, N-methylpyrrolidone, dimethyl acetamide (DMA), dimethylsulfoxide (DMSO), dimethyl formamide, glycerol formal, ethoxy diglycol, triethylene glycol dimethyl ether, triacetin, diacetin, corn oil, acetyl triethyl citrate (ATC), ethyl lactate, polyglycolated capryl glycerides butyrolactone, dimethyl isosorbide, benzyl alcohol, ethanol, isopropyl alcohol, propylene glycol, purified water. The preferred co-solvent is glycerol or glycerin. The amount of glycerin/glycerol is present in amount between about 40% w/v to about 50% w/v, more preferably between about 45% w/v to about 48% w/v.

In another embodiment, preservatives used in the composition of the present invention are selected from but are not limited to, benzyl benzoates, benzoic acid, benzyl alcohol, benzalkonium chloride, N-cetyl-N,N,N-trimethylammonium bromide (Cetrimide, Merck), chlorhexidine, chlorobutanol, chlorocresol, iminourea, parabens such as methyl-, ethyl-, propyl- or butylparaben, sodium methylparaben, sodium propylparaben, potassium sorbate, sodium benzoate, sodium propionate, phenol, phenoxyethanol, phenylethyl alcohol, phenylmercuric acetate, phenyl-mercuric borate, phenylmercuric nitrates, sorbic acid or thiomersal (sodium ethylmercurithiosalicylate), sepicide, tween 80, bithional, butyl p-hydroxy benzoate, p-chloro-m-xylenol, dehydro acetic acid, methyl-p-hydroxybenzoate, propyl-p-hydroxybenzoate and mixtures thereof. Methylparaben, propylparaben, sodium methylparaben and sodium propylparaben are the preferred preservatives. The preservatives may be present from about 0.1% w/v to about 0.5% w/v, more preferably between about 0.15% w/v to about 0.45% w/v.

Further, the present invention provides a process for preparation of a stable pharmaceutical compositions comprising;
a) Nimodipine, as an active ingredient;
b) Polyethylene glycol and alcohol as solvent;
c) Co-solvent;
d) purified water; and
e) one or more pharmaceutically acceptable excipient;
   Wherein the process comprises:
(a) Preparation of glycerin solution by heating at 80-90° C. and add methyl paraben under continuous stirring to get clear solution;
(b) Preparation of nimodipine solution by using PEG-400 and ethanol as solvent;
(c) Mixing (b) into (a) with addition of water;
(d) Filter the bulk solution (c) through 3.0μ (Polypropylene) filter; and
(e) Transfer the filtered solution into storage vessel.

In an embodiment, the present invention provides a stable aqueous pharmaceutical composition in a liquid oral solution dosage form comprising about 3 mg/10 mL of nimodipine to about 12 mg/10 ml of nimodipine, more preferably about 4 mg/10 ml of nimodipine to about 10 mg/ml of nimodipine.

In another embodiment, the present invention provides a stable aqueous pharmaceutical composition in a liquid oral solution dosage form comprising about 3 mg/mL of nimodipine.

In another embodiment, the present invention provides a stable aqueous pharmaceutical composition in a liquid oral solution dosage form comprising about 6 mg/mL of nimodipine.

In another embodiment, the present invention provides a stable aqueous pharmaceutical composition in a liquid oral solution dosage form comprising about 12 mg/mL of nimodipine.

In another embodiment, the invention relates to a stable aqueous solution pharmaceutical composition for liquid oral solution administration comprising:

(i) Nimodipine,

(ii) Solvent,

(iii) Co-solvent,

(iv) Purified water and

(v) One or more excipients

In another embodiment, the invention relates to a stable aqueous solution pharmaceutical composition for liquid oral solution administration comprising:

(vi) Nimodipine,

(vii) Solvent consisting of PEG 400 and Alcohol,

(viii) Glycerin,

(ix) water and

(x) Preservative.

In another embodiment, the present invention provides a stable aqueous solution pharmaceutical composition in a liquid oral solution dosage form wherein the dosage form comprises:

about 0.1-0.60% w/v nimodipine,

about 65-70% w/v polyethylene glycol 400,

about 45.17-47.55% w/v glycerin,

about 2.30-4.68% w/v purified water,

about 0.35-0.45% w/v absolute alcohol and

about 0.20-0.30% w/v methyl paraben.

In another embodiment, the present invention provides a stable aqueous pharmaceutical composition in a liquid oral solution dosage form wherein the liquid oral solution dosage form comprises of:

0.60% w/v Nimodipine,

66% w/v polyethylene glycol 400,

0.20% w/v methyl paraben,

0.35% w/v absolute alcohol,

2.3-4.68% w/v purified water and

45.17-47.55% w/v glycerin.

In another embodiment, the present invention provides a stable aqueous pharmaceutical composition in a liquid oral solution dosage form wherein the liquid oral solution dosage form consisting of:

about 0.60% w/v Nimodipine,

about 66% w/v polyethylene glycol 400,

about 0.20% w/v methyl paraben,

about 0.35% w/v absolute alcohol,

about 2.3% w/v purified water and

about 45.17% w/v glycerin

In another embodiment, the present invention provides a stable aqueous pharmaceutical composition in a liquid oral solution dosage form wherein the liquid oral solution dosage form consisting essentially of:

about 0.60% w/v Nimodipine,

about 66% w/v polyethylene glycol 400,

about 0.20% w/v methyl paraben,

about 0.35% w/v absolute alcohol,

about 2.3% w/v purified water and

about 45.17% w/v glycerin

In another embodiment, a stable aqueous solution composition suitable for oral administration consisting of:
1. Nimodipine at a concentration of about 3 mg/ml to about 12 mg/ml;
2. a pharmaceutically acceptable solvent system consisting of Polyethylene glycol and alcohol in a concentration of about 500 mg/ml to about 700 mg/ml and about 1 mg/ml to about 5 mg/ml respectively;
3. glycerin at a concentration of about 400 mg/ml to about 500 mg/ml;
4. water at a concentration of about 20 mg/ml to about 50 mg/ml; and
5. methyl paraben at a concentration of about 1 mg/ml to about 5 mg/ml.
   In another embodiment, a stable aqueous solution composition suitable for oral administration consisting of:
1. Nimodipine at a concentration of 6 mg/ml;
2. a pharmaceutically acceptable solvent system consisting of Polyethylene glycol and alcohol in a concentration of about 670 mg/ml and about 4 mg/ml respectively;
3. glycerin at a concentration of about 480 mg/ml;
4. water at a concentration of about 25 mg/ml; and
5. methyl paraben at a concentration of about 3 mg/ml.

An aspect of the present invention provides a stable pharmaceutical composition of nimodipine indicated for the improvement of neurological outcome by reducing the incidence and severity of ischemic deficits in adult patients with subarachnoid hemorrhage (SAH). Another aspect of the present invention further provides a process of preparation of a stable pharmaceutical composition of nimodipine oral solution.

It should be appreciated that the invention can be embodied/aspects in different forms and should not be construed as limited to the embodiments set forth herein. Rather, these embodiments are provided so that this disclosure will be thorough and complete, and will convey the scope of the invention to those skilled in the art. Other features and embodiments of the invention will become apparent from the following examples, which are given for illustration of the invention rather than for limiting its intended scope.

The following examples are intended to illustrate the scope of the present invention in all its aspects but not to limit it thereto.

### Example 1

| Sr. No. | Ingredient | Std. Qty. in mg/10 mL |
|---|---|---|
| 1 | Nimodipine | 60.00 |
| 2 | Polyetylene Glycol 400 | 5600.00 |
| 3 | Methyl paraben | 20.00 |
| 4 | Absolute Alcohol | 45.00 |
| 5 | Purified water | 300.00 |
| 6 | Glycerin | 4755.00 |

| | |
|---|---|
| Total | Qs to 10 mL |

Qs: Quantity sufficient

### Manufacturing Process

The active and inactive ingredients were dispensed and shifted to a mixing vessel. The glycerin part I solution was prepared in mixing vessel by heating at temperature 80-90° C. and add measured amount of methyl paraben under continuous stirring, cooled solution at room temperature under stirring. The nimodipine solution was prepared by using polyethylene glycol 400 and absolute alcohol was added into active mixing vessel and Nimodipine was added into it under continuous stirring. The prepared nimodipine solution was added to the prepared glycerin part I solution in mixing vessel and add purified water into the mixing vessel and stirred. The final weight adjustment of the bulk solution in mixing vessel was done by using glycerin part II. After weight adjustment, stirred the bulk solution in mixing vessel found to be clear light-yellow coloured solution. The final solution was filtered and filled into pharmaceutically acceptable containers.

### Example 2

| Sr. No. | Ingredient | % W/V |
|---|---|---|
| 1 | Nimodipine | 0.1-0.60 |
| 2 | Polyetylene Glycol 400 | 65-70 |
| 3 | Methyl paraben | 0.20-0.30 |
| 4 | Absolute Alcohol | 0.35-0.45 |
| 5 | Purified water | 2.30-4.68 |
| 6 | Glycerin | 45.17-47.55 |

Std. Qty. in mg/10 mL

Qs: Quantity sufficient

### Manufacturing Procedure:

The active and inactive ingredient were dispensed and sifted to a mixing vessel. The glycerin part I solution was prepared in mixing vessel by heating at temperature 80-90° C. and add measured amount of methyl paraben under continuous stirring, cooled solution at room temperature under stirring. The nimodipine solution was prepared by using polyethylene glycol 400 and absolute alcohol was added into active mixing vessel and Nimodipine was added into it under continuous stirring. The prepared nimodipine solution were added to prepared glycerin part I solution in mixing vessel and add purified water into the mixing vessel and stirred. The final weight adjustment of the bulk solution in mixing vessel was done by using glycerin part II. After weight adjustment, stirred the bulk solution in mixing vessel found to be clear light-yellow coloured solution. The final solution was filtered and filled into pharmaceutically acceptable containers.

The distinct formulations of the invention exemplified in example 2 were evaluated for stability conditions at 25° C.±2° C. and 60% RH±5% RH, 40° C.±2° C. and 75% RH±5% RH for initial and 3M (months).

The stability results are provided in the table below:

A. Stability Study: Example 1 at 25° C.±2° C. and 60% RH±5% RH, 40° C.±2° C. and 75% RH±5% RH for initial and 3M (months).

TABLE 1

| | | 5 mL | 10 mL | 5 mL | 10 mL | | | |
|---|---|---|---|---|---|---|---|---|
| | | PFS | PFS | PFS | PFS | 237 mL Bottle | | |
| Related | | 25° C. ± 2° C./ | | 40° C. ± 2° C./ | | | 25° C./ | 40° C./ |
| substances | Initial | 60% RH ± 5% RH | | 75% RH ± 5% RH | | Initial | 60%RH | 75% RH |
| Assay | 100.5 | 99.4 | 98.9 | 99.0 | 99.3 | 101.4 | 101.1 | 99.7 |
| (Nimodipine | | | | | | | | |
| 90-100%) | | | | | | | | |
| Assay (Methyl | 103.7 | 99.4 | 98.6 | 101.3 | 99.3 | 99.8 | 100.4 | 99.5 |
| Paraben | | | | | | | | |
| 180-20%) | | | | | | | | |
| Water Content | 2.71 | 2.64 | 2.65 | 2.68 | 2.67 | 2 | 2 | 2 |
| (w/w by KF; | | | | | | | | |
| 1-4%) | | | | | | | | |
| Nimodipine | 0.02 | 0.07 | 0.08 | 0.33 | 0.33 | 0.06 | 0.04 | 0.35 |
| Related | | | | | | | | |
| compound A | | | | | | | | |
| (Impurity A)- | | | | | | | | |
| NMT 0.5% | | | | | | | | |
| Any | 0.03 | 0.06 | 0.07 | 0.11 | 0.13 | 0.04 | 0.03 | 0.15 |
| Unspecified | | | | | | | | |
| degradation | | | | | | | | |
| product-NMT | | | | | | | | |
| 0.2% | | | | | | | | |
| Total | 0.05 | 0.19 | 0.23 | 0.63 | 0.55 | 0.1 | 0.1 | 0.5 |
| degradation | | | | | | | | |
| product-NMT | | | | | | | | |
| 1.0% | | | | | | | | |

All the physical and chemical parameters were found satisfactory and the initial and 3M stability data at 25° C.±2° C. and 60% RH±5% RH, 40° C.±2° C. and 75% RH±5% RH was found to be satisfactory.
The distinct formulations of the invention exemplified in example 1 and 2 were evaluated for stability conditions at 25° C.±2° C. and 60% RH±5% RH, 30° C.±2° C. and 65% RH±5% RH for initial and 12M (months).
The stability results are provided in table below:

B. Stability Study: Example 1 at 25° C.±2° C. and 60% RH±5% RH, 30° C.±2° C. and 65% RH±5% RH for initial and 12M (months).

TABLE 2

| | | 5 mL PFS | | 10 mL PFS | | 237 mL Bottle | | |
|---|---|---|---|---|---|---|---|---|
| | | 25° C./ | 30° C./ | 25° C./ | 30° C./ | | 25° C./ | 30° C./ |
| Related | | 60% RH | 65% RH | 60% RH | 65% RH | | 60% RH | 65% RH |
| substances | Initial | 12M | 12M | 12M | 12M | Initial | 12M | 12M |
| Assay | 101.7 | 100.9 | 100.3 | 100.5 | 99.1 | 101.4 | 100.6 | 98.6 |
| (Nimodipine | | | | | | | | |
| 90-100%) | | | | | | | | |
| Assay (Methyl | 100.8 | 101.3 | 101 | 101 | 101.2 | 99.8 | 104 | 101.2 |
| Paraben 80- | | | | | | | | |
| 120%) | | | | | | | | |
| Water Content | 3 | 3 | 1.21 | 3 | 3 | 2.484 | 2 | 1.19 |
| (w/w by KF; | (2.50) | | | | | | | |
| 1-4%) | | | | | | | | |
| Nimodipine | BLQ | 0.21 | 0.43 | 0.2 | 0.43 | 0.06 | 0.25 | 0.44 |
| Related | (0.044%) | | | | | | | |
| compound A | | | | | | | | |
| (Impurity A)- | | | | | | | | |
| NMT 0.5% | | | | | | | | |
| Any Unspecified | 0.02 | 0.06 | 0.08 | 0.12 | 0.15 | 0.04 | 0.11 | 0.16 |
| degradation | | | | | | | | |
| product-NMT | | | | | | | | |
| 0.2% | | | | | | | | |
| Total degradation | 0 | 0.3 | 0.06 | 0.4 | 0.7 | 0.1 | 0.5 | 0.7 |
| product-NMT | | | | | | | | |
| 1.0% | | | | | | | | |

All the physical and chemical parameters were found satisfactory and the initial and 12M stability data at 25° C.±2° C. and 60% RH±5% RH, 30° C.±2° C. and 65% RH±5% RH was found to be satisfactory.

We claim:

1. A stable aqueous solution composition suitable for oral administration comprising:
a. Nimodipine;
b. a pharmaceutically acceptable solvent system consisting of Polyethylene glycol and alcohol;
c. a pharmaceutically acceptable Co-solvent;
d. water, and
e. One or more pharmaceutically acceptable excipients.

2. A stable aqueous solution composition as in claim 1, wherein Nimodipine is present at a concentration of about 3 mg/ml to about 12 mg/ml.

3. A stable aqueous solution composition as in claim 1, wherein the solvent system consisting of Polyethylene glycol and alcohol is present at a concentration of about 500 mg/ml to about 700 mg/ml and about 1 mg/ml to about 5 mg/ml respectively.

4. A stable aqueous solution composition as in claim 1, wherein the co-solvent selected is from the group consisting of glycerin, dimethylsulfoxide, N-methylpyrrolidone, dimethyl acetamide (DMA), dimethylsulfoxide (DMSO), dimethyl formamide, glycerol formal, ethoxy diglycol, triethylene glycol dimethyl ether, triacetin, diacetin, corn oil, acetyl triethyl citrate (ATC), ethyl lactate, polyglycolated capryl glycerides butyrolactone, dimethyl isosorbide, benzyl alcohol, ethanol, isopropyl alcohol and propylene glycol or a combination thereof.

5. A stable aqueous solution composition as in claim 4, wherein the co-solvent is glycerin and is present at a concentration of about 400 mg/ml to about 500 mg/ml.

6. A stable aqueous solution composition as in claim 1, wherein the pharmaceutical excipient is a preservative selected from the group consisting of benzyl benzoates, benzoic acid, benzyl alcohol, benzalkonium chloride, parabens such as methyl-, ethyl-, propyl- or butylparaben, sodium methylparaben, sodium propylparaben, potassium sorbate, sodium benzoate, sodium propionate, phenol, phenoxyethanol, phenylethyl alcohol, tween 80, bithional, butyl p-hydroxy benzoate, p-chloro-m-xylenol, dehydro acetic acid, methyl-p-hydroxybenzoate, propyl-p-hydroxybenzoate and mixtures thereof.

7. A stable aqueous solution composition as in claim 6, wherein the preservative is methyl paraben and is present at a concentration of about 1 mg/ml to about 5 mg/ml.

8. A stable aqueous solution composition as in claim 1, wherein the water is present at a concentration of about 20 mg/ml to about 50 mg/ml.

9. A stable aqueous solution composition suitable for oral administration consisting of:
a. Nimodipine at a concentration of about 3 mg/ml to about 12 mg/ml;
b. a pharmaceutically acceptable solvent system consisting of Polyethylene glycol and alcohol in a concentration of about 500 mg/ml to about 700 mg/ml and about 1 mg/ml to about 5 mg/ml respectively;
c. glycerin at a concentration of about 400 mg/ml to about 500 mg/ml;
d. water at a concentration of about 20 mg/ml to about 50 mg/ml; and
e. methyl paraben at a concentration of about 1 mg/ml to about 5 mg/ml.

10. A stable aqueous solution composition suitable for oral administration consisting of:
a. Nimodipine at a concentration of 6 mg/ml;
b. a pharmaceutically acceptable solvent system consisting of Polyethylene glycol and alcohol in a concentration of about 670 mg/ml and about 4 mg/ml respectively;
c. glycerin at a concentration of about 480 mg/ml;
d. water at a concentration of about 25 mg/ml; and
e. methyl paraben at a concentration of about 3 mg/ml.
